SEQUENCE LISTING (4 sequences)

SEQ ID NO: 1 - RNA, 22 nt, Homo sapiens
  source 1..22: mol_type = genomic RNA
tagcttatcagactgatgttga

SEQ ID NO: 2 - RNA, 72 nt, Homo sapiens
  source 1..72: mol_type = genomic RNA
tgtcgggtagcttatcagactgatgttgactgttgaatctcatggcaacaccagtcgatgggctgtctgaca

SEQ ID NO: 3 - DNA, 19 bp, synthetic construct
  modified_base 13: mod_base = OTHER; note = (S)-constrained ethyl-uracil
  source 1..19: mol_type = other DNA
  modified_base 1: mod_base = OTHER; note = 2'-O-Methoxyethyl-adenosine
  modified_base 19: mod_base = OTHER; note = 2'-O-Methoxyethyl-adenosine
  modified_base 2: mod_base = OTHER; note = (S)-constrained ethyl-cytosine
  modified_base 5: mod_base = OTHER; note = (S)-constrained ethyl-cytosine
  modified_base 9: mod_base = OTHER; note = (S)-constrained ethyl-cytosine
  modified_base 17: mod_base = OTHER; note = (S)-constrained ethyl-cytosine
  modified_base 1..19: mod_base = OTHER; note = each internucleoside linkage is a phosphorothioate internucleoside linkage
acatcagtctgataagcta

SEQ ID NO: 4 - DNA, 18 bp, synthetic construct
  modified_base 13: mod_base = OTHER; note = (S)-constrained ethyl-uracil
  source 1..18: mol_type = other DNA
  modified_base 2: mod_base = OTHER; note = (S)-constrained ethyl-cytosine
  modified_base 1: mod_base = OTHER; note = 2'-O-Methoxyethyl-adenosine
  modified_base 5: mod_base = OTHER; note = (S)-constrained ethyl-cytosine
  modified_base 9: mod_base = OTHER; note = (S)-constrained ethyl-cytosine
  modified_base 17: mod_base = OTHER; note = (S)-constrained ethyl-cytosine
  modified_base 1..18: mod_base = OTHER; note = each internucleoside linkage is a phosphorothioate internucleoside linkage
acatcagtctgataagct

(19) United States
(12) Patent Application Publication
(10) Pub. No.: US 2024/0115598 A1
(43) Pub. Date: Apr. 11, 2024

(54) Methods for Treatment of Alport Syndrome

(71) Applicant: Sanofi, Paris (FR)

(72) Inventor: Timothy Wright, San Diego, CA (US)

(73) Assignee: Sanofi, Paris (FR)

(21) Appl. No.: 18/446,743

(22) Filed: Aug. 9, 2023

Related U.S. Application Data
(63) Continuation of application No. 17/474,264, filed on Sep. 14, 2021, now abandoned.
Application No. 17/474,264 is a continuation of application No. 16/607,971, filed on Oct. 24, 2019, now abandoned.
(60) Provisional application No. 62/501,699, filed on May 4, 2017.

(51) Int. Cl. A61K 31/712; A61K 9/00; A61K 48/00; A61P 13/12; C12N 15/113

(52) U.S. Cl. CPC A61K 31/712; A61K 9/0019; A61K 48/00; A61P 13/12; C12N 15/113; C12N 2310/11; C12N 2310/141

(57) ABSTRACT

Provided herein are methods for the treatment of Alport syndrome, using a modified oligonucleotide targeted to miR-21. In certain embodiments, the modified oligonucleotide targeted to miR-21 improves kidney function and/or reduces fibrosis in subjects having Alport syndrome. In certain embodiments, administration of the modified oligonucleotide targeted to miR-21 delays the onset of end-stage renal disease in a subject having Alport syndrome. In certain embodiments, the modified oligonucleotide targeted to miR-21 delays the need for dialysis or kidney transplant in a subject having Alport syndrome.

### CROSS-REFERENCE TO RELATED APPLICATIONS

This application is continuation of U.S. application Ser. No. 17/474,264, filed Sep. 14, 2021, which is a continuation of U.S. application Ser. No. 16/607,971, filed Oct.24, 2019, which is a national phase entry pursuant to 35 U.S.C. § 371 of International Application No. PCT/US2018/031094, filed May 4, 2018, which claims the benefit of priority of U.S. Provisional Application No. 62/501,699, filed May 4, 2017, which is incorporated by reference herein in its entirety for any purpose.

### SEQUENCE LISTING

The present application contains a Sequence Listing which has been submitted electronically in XML format. Said XML copy, created on Jul. 28, 2023 is named “2023-07-26-01232-0037-02US-ST26.xml” and is 12,840 bytes in size. The information in the electronic format of the sequence listing is incorporated herein by reference in its entirety.

### FIELD OF INVENTION

Provided herein are methods and compositions for the treatment of Alport syndrome.

### BACKGROUND

Type IV collagen, a major component of the basement membrane, is a family of six alpha chains: alpha-1 collagen (Type IV), alpha-2 collagen (Type IV), alpha-3 collagen (Type IV), alpha-4 collagen (Type IV), alpha-5 collagen (Type IV), and alpha-6 collagen (Type IV). The alpha-3, alpha-4 and alpha-6 chains of collagen IV are fundamental components of the collagen network of the glomerular basement membrane (GBM), which performs the critical function of filtration of blood by the kidney.

Alport syndrome is an inherited form of kidney disease in which an abnormal type of glomerular basement membrane (GBM) is produced, leading to interstitial fibrosis, glomerular sclerosis and eventual loss of kidney function. The disease is also frequently characterized by hearing defects and ocular anomalies. Alport syndrome is caused by a mutation in Col4a3, Col4a4, or Col4a5, which encode the alpha3(IV), alpha4(IV), and alpha5(IV) chains of type IV collagen, respectively. Mutations in the Col4a5 gene on the X chromosome cause the X-linked form of Alport syndrome, which accounts for 85% of all cases of the disease. An autosomal recessive form is due to inheritance of mutations in each copy of either Col4a3 or Col4a4, each of which is located on chromosome 2.

The rare autosomal dominant form is due to inheritance of a dominant-negative mutation in either the Col4a3 or Col4a4 gene. The X-linked form is more severe in males than in females, with most cases in males progressing to end-stage renal disease (ESRD) . The autosomal form is of similar severity in males and females. Most cases of the disease are due to an inherited mutation, but some cases are due to a de novo mutation in one of the Col4aA genes.

### SUMMARY

Embodiment 1. A method for treating Alport syndrome comprising administering to a subject having Alport syndrome two or more doses of a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, and wherein a dose of 1.5 mg/kg is administered at a frequency of two weeks between doses.

Embodiment 2. The method of embodiment 1, wherein the dose is delivered in a pharmaceutically acceptable diluent.

Embodiment 3. The method of embodiment 2, wherein the pharmaceutically acceptable diluent is a saline solution.

Embodiment 4. The method of embodiment 3, wherein the saline solution is a 0.3% sodium chloride solution.

Embodiment 5. The method of any one of embodiments 2 to 4, wherein the concentration of the modified oligonucleotide in the pharmaceutically acceptable diluent is at least 110 mg/mL.

Embodiment 6. The method of any one of embodiments 1 to 5, wherein the dose is a single bolus injection of 110 mg/mL of the modified oligonucleotide.

Embodiment 7. The method of any one of embodiments 1 to 6, wherein the pharmaceutical composition is administered as a subcutaneous injection.

Embodiment 8. The method of embodiment 7, wherein the subcutaneous injection is administered in the anterior abdominal wall of the subject.

Embodiment 9. The method of any one of embodiments 1 to 8, comprising selecting a subject who has been diagnosed with Alport syndrome by clinical, histopathologic, and/or genetic criteria.

Embodiment 10. The method of any of embodiments 1 to 9, wherein the subject has an estimated glomerular filtration rate of 30 ml/min/1.73 m² prior to receiving the first dose of the modified oligonucleotide.

Embodiment 11. The method of embodiment 15, wherein the subject has an estimated glomerular filtration rate (eGFR) between 45 and 90 ml/min/1.73 m² prior to receiving the first dose of the modified oligonucleotide.

Embodiment 12. The method of any one of embodiments 1 to 11, wherein the estimated glomerular filtration rate of the subject is declining at a rate ml/min/1.73 m²/year prior to receiving the first dose of the modified oligonucleotide.

Embodiment 13. The method of any one of embodiments 1 to 12, wherein the subject is male, has been diagnosed with X-linked Alport syndrome, and is between 18 and 30 years of age.

Embodiment 14. The method of any one of embodiments 1 to 13, wherein the subject has proteinuria of greater than 300 milligrams of protein per gram of creatinine prior to receiving the first dose of the modified oligonucleotide.

Embodiment 15. The method of any one of embodiments 1 to 14, wherein the subject, following administration of the modified oligonucleotide, experiences an improvement in one or more parameters associated with Alport syndrome selected from the group consisting of:
a. estimated glomerular filtration rate;
b. rate of decline in estimated glomerular filtration rate; and
c. quality of life using the Short Form 36 Health Survey®.

Embodiment 16. The method of any one of embodiments 1 to 15, wherein the subject, following administration of the modified oligonucleotide, exhibits an improvement in one or more renal biomarkers selected from the group consisting of:
a. miR-21 in biopsy tissue;
b. blood urea nitrogen;
c. urine protein/albumin ratio;
d. urine albumin/creatine ratio;
e. creatinine;
f. urine podocyturia;
g. kidney injury molecule-1;
h. beta-2 microglobulin;
i. clusterin;
j. cystatin C;
k. asymmetric dimethylarginine;
l. transforming growth factor-beta;
m. connective tissue growth factor; and
n. neutrophil gelatinase-associated lipocalin.

Embodiment 17. The method of any one of embodiments 1 to 16, wherein one or more of creatinine, cystatin C, kidney injury molecule-1, beta-2 microglobulin, and/or clusterin is measured in a blood sample of the subject.

Embodiment 18. The method of any one of embodiments 1 to 16, wherein one or more of creatinine, cystatin C, kidney injury molecule-1, beta-2 microglobulin, and/or clusterin is measured in a urine sample of the subject.

Embodiment 19. The method of any one of embodiments 1 to 18, wherein the subject has been treated with an angiotensin II converting enzyme (ACE) inhibitor for at least 30 days prior to receiving the first dose of the oligonucleotide.

Embodiment 20. The method of any one of embodiments 1 to 19, wherein the subject has been treated with an angiotensin II receptor blocker (ARB) for at least 30 days prior to receiving the first dose of the oligonucleotide.

Embodiment 21. The method of embodiment 19 wherein the angiotensin II converting enzyme (ACE) inhibitors is selected from captopril, enalapril, lisinopril, benazepril, quinapril, fosinopril, and ramipril.

Embodiment 22. The method of embodiment 20 wherein the angiotensin II receptor blockers (ARB) is selected from candesartan, irbesartan, olmesartan, losartan, valsartan, telmisartan, and eprosartan.

Embodiment 23. The method of any one of embodiments 1 to 22, wherein at least 24 doses are administered to the subject.

Embodiment 24. A method for treating Alport syndrome in a subject, the method comprising:
a. selecting a subject who has been diagnosed with Alport syndrome using clinical, histopathologic, and/or genetic criteria;
b. administering to the subject two or more doses of a pharmaceutical composition comprising a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, wherein the dose of the modified oligonucleotide is 1.5 mg/kg and wherein the doses are administered with a frequency of two weeks between doses,
c. wherein the subject, following administration of the pharmaceutical composition, exhibits an improvement in one or more AS associated parameters selected from:
   i. estimated glomerular filtration rate (eGFR);
   ii. rate of decline of eGFR; and
   iii. quality of life (QOL) as measured by the Short Form 36 Health Survey®.

Embodiment 25. A method for treating Alport syndrome in a subject, the method comprising:
a. selecting a subject who has been diagnosed with Alport syndrome using clinical, histopathologic, and/or genetic criteria, wherein the subject has:
   i. an estimated glomerular filtration rate of at least 30 ml/min/1.73 m²;
   ii. a decline in the rate of estimated glomerular filtration rate of ml/min/1.73 m²/year;
   iii. proteinuria greater equal to or greater than 300 mg protein/g creatinine; and
   iv. been treated with a stable dosing regimen of an ACE inhibitor and/or an ARB for at least 30 days;
b. administering to the subject two or more doses of a pharmaceutical composition comprising a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, wherein the dose of the modified oligonucleotide is 1.5 mg/kg and wherein the doses are administered with a frequency of two weeks between doses,
c. wherein the subject, following administration of the pharmaceutical composition, exhibits an improvement in one or more parameters associated with Alport syndrome selected from:
   i. estimated glomerular filtration rate (eGFR);
   ii. rate of decline of eGFR; and
   iii. quality of life (QOL) as measured by the Short Form 36 Health Survey®.

Embodiment 26. A method for reducing decline in renal function over time in a subject with Alport syndrome, the method comprising:
a. selecting a subject diagnosed with Alport syndrome confirmed by clinical, histopathologic, and/or genetic criteria, wherein the subject has:
   i. an estimated glomerular filtration rate of at least 30 ml/min/1.73 m²;
   ii. a decline in the rate of estimated glomerular filtration rate of ml/min/1.73 m²/year;
   iii. proteinuria greater equal to or greater than 300 mg protein/g creatinine; and
   iv. been treated with a stable dosing regimen of an ACE inhibitor and/or an ARB for at least 30 days;
b. administering to the subject two or more doses of a pharmaceutical composition comprising a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, wherein the dose of the modified oligonucleotide is 1.5 mg/kg and wherein the doses are administered with a frequency of two weeks between doses,
c. wherein the subject, following administration of the pharmaceutical composition, exhibits an improvement in one or more Alport syndrome associated parameters selected from:
   i. estimated glomerular filtration rate (eGFR);
   ii. rate of decline of eGFR; and
   iii. quality of life (QOL) as measured by the Short Form 36 Health Survey®. Embodiment 27. The method of any one of embodiments 1 to 26, wherein the modified oligonucleotide has the structure:

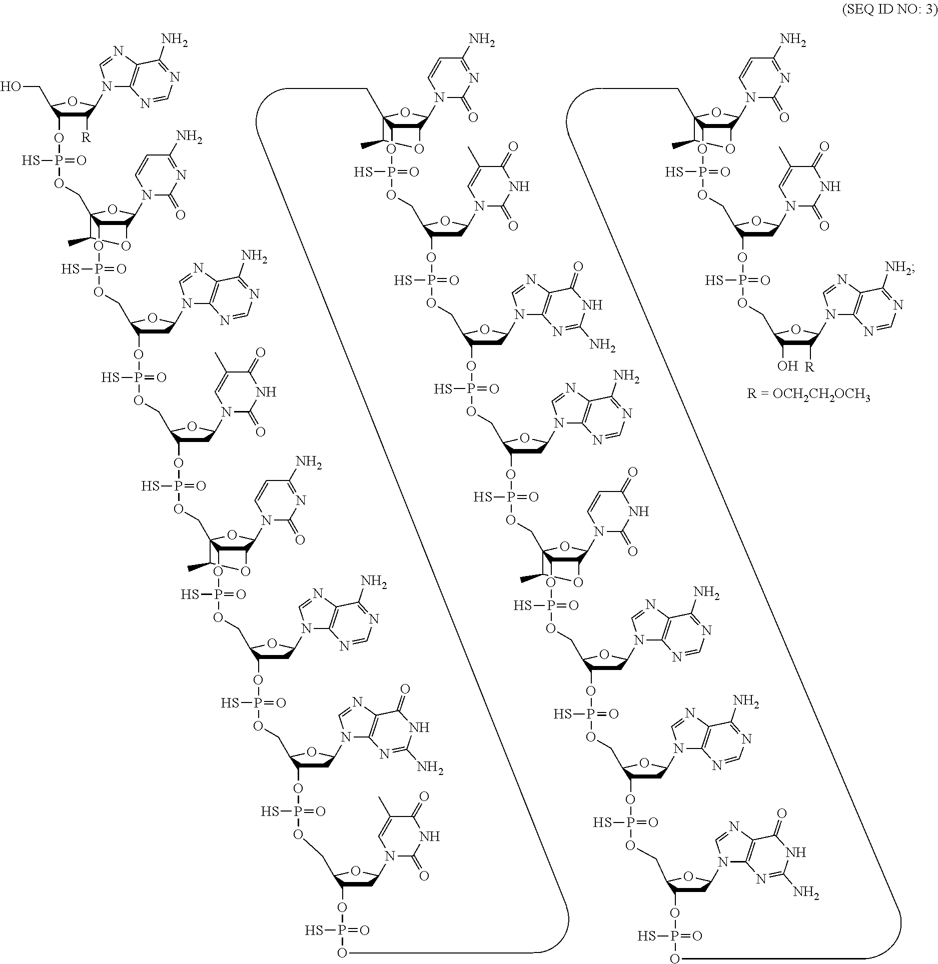

or a pharmaceutically acceptable salt thereof.

Embodiment 28. The method of embodiment 27, wherein the modified oligonucleotide is present as a pharmaceutically acceptable salt of the structure.

Embodiment 29. The method of embodiment 28, wherein the modified oligonucleotide is present as a sodium salt of the structure.

Embodiment 30. The method of any one of embodiments 1 to 29, wherein the modified oligonucleotide has the structure:

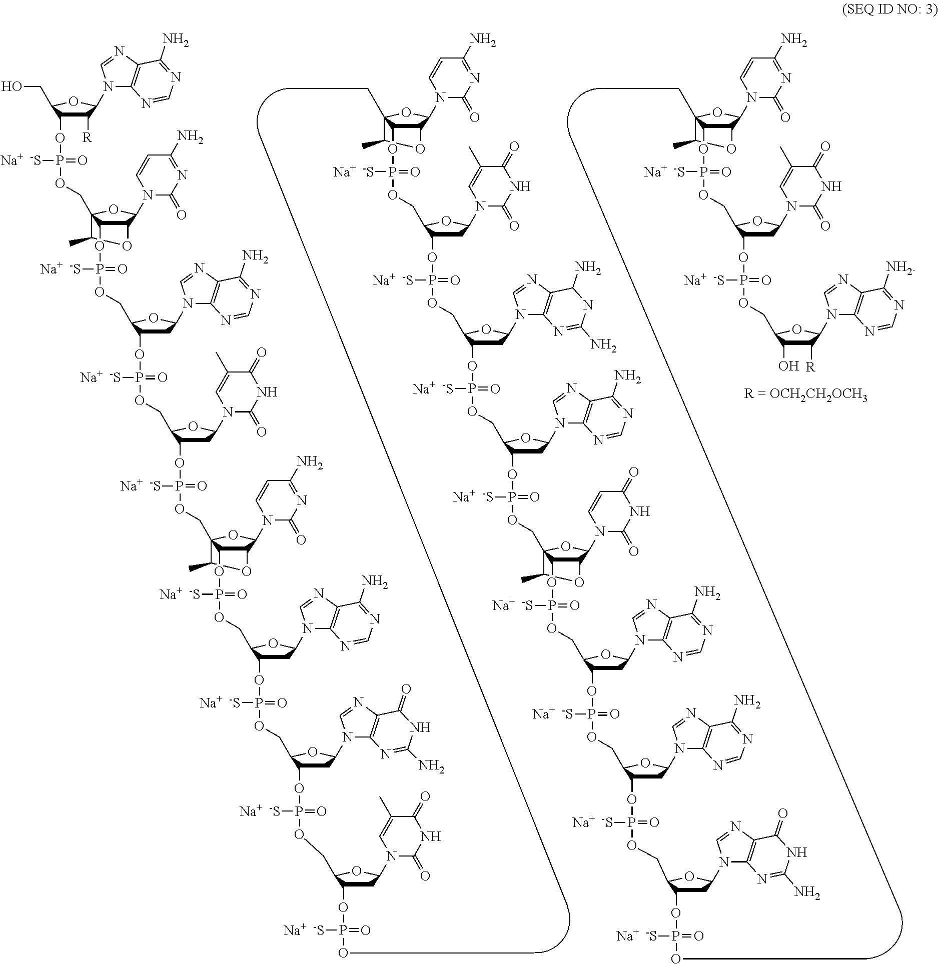

Embodiment 31. The method of any one of embodiments 10 to 30, wherein the estimated glomerular filtration rate is calculated using the Chronic Kidney Disease Epidemiology Collaboration (CKD-EPI) creatinine equation.

Embodiment 32. The method of any one of embodiments 10 to 30, wherein the estimated glomerular filtration rate is calculated using the Chronic Kidney Disease Epidemiology Collaboration (CKD-EPI) creatinine-cystatin C equation.

### DETAILED DESCRIPTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sangvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can change, but equivalent information can be found by searching the internet. Reference thereto evidences the availability and public dissemination of such information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Alport syndrome” means an inherited form of kidney disease in which an abnormal level of glomerular basement membrane (GBM) is produced, leading to interstitial fibrosis, glomerular sclerosis and eventual loss of kidney function. The disease is also frequently characterized by hearing defects and ocular anomalies.

“Hematuria” means the presence of red blood cells in the urine.

“Albuminuria” means the presence of excess albumin in the urine, and includes without limitation, normal albuminuria, high normal albuminuria, microalbuminuria and macroalbuminuria. Normally, the glomerular filtration permeability barrier, which is composed of podocyte, glomerular basement membrane and endothelial cells, prevents serum protein from leaking into urine. Albuminuria may reflect injury of the glomerular filtration permeability barrier. Albuminuria may be calculated from a 24-hour urine sample, an overnight urine sample or a spot-urine sample.

“High normal albuminuria” means elevated albuminuria characterized by (i) the excretion of to <30 mg of albumin into the urine per 24 hours and/or (ii) an albumin/creatinine ratio of 1.25 to <2.5 mg/mmol (or 10 to <20 mg/g) in males or 1.75 to <3.5 mg/mmol (or 15 to <30 mg/g) in females.

“Microalbuminuria” means elevated albuminuria characterized by (i) the excretion of 30 to 300 mg of albumin into the urine per 24 hours and/or (ii) an albumin/creatinine ratio of 2.5 to <25 mg/mmol (or 20 to <200 mg/g) in males or 3.5 to <35 mg/mmol (or 30 to <300 mg/g) in females.

“Macroalbuminuria” means elevated albuminuria characterized by the excretion of more than 300 mg of albumin into the urine per 24 hours and/or (ii) an albumin/creatinine ratio of >25 mg/mmol (or >200 mg/g) in males or >35 mg/mmol (or >300 mg/g) in females.

“Albumin/creatinine ratio” means the ratio of urine albumin (mg/dL) per urine creatinine (g/dL) and is expressed as mg/g. In certain embodiments, albumin/creatinine ratio may be calculated from a spot-urine sample and may be used as an estimate of albumin excretion over a 24 hour period.

“Glomerular filtration rate (GFR)” means the flow rate of filtered fluid through the kidney and is used as an indicator of kidney function in a subject. In certain embodiments, a subject's GFR is determined by calculating an estimated glomerular filtration rate. In certain embodiments, a subject's GFR is directly measured in the subject, using the inulin method.

“Estimated glomerular filtration rate (eGFR) means ameasurement of how well the kidneys are filtering creatinine, and is used to approximate glomerular filtration rate. As the direct measurement of GFR is complex, eGFR is frequently used in clinical practice. Normal results may range from⁹⁰-120 mL/min/1.73 m². Levels below 60 mL/min/1.73 m² for 3 or more months may be an indicator chronic kidney disease. Levels below 15 mL/min/1.73 m² may be an indicator of kidney failure.

“Proteinuria” means the presence of an excess of serum proteins in the urine. Proteinuria may be characterized by the excretion of >250 mg of protein into the urine per 24 hours and/or a urine protein to creatinine ratio of ≥0.20 mg/mg. Serum proteins elevated in association with proteinuria include, without limitation, albumin.

“Blood urea nitrogen” or “BUN” means a measure of the amount of nitrogen in the blood in the form of urea. The liver produces urea in the urea cycle as a waste product of the digestion of protein, and the urea is removed from the blood by the kidneys. Normal human adult blood may contain between 7 to 21 mg of urea nitrogen per 100 ml (7-21 mg/dL) of blood. Measurement of blood urea nitrogen is used as an indicator of renal health. If the kidneys are not able to remove urea from the blood normally, a subject's BUN rises.

“End stage renal disease (ESRD)” means the complete or almost complete failure of kidney function.

“Impaired kidney function” means reduced kidney function, relative to normal kidney function.

“Fibrosis” means the formation or development of excess fibrous connective tissue in an organ or tissue. In certain embodiments, fibrosis occurs as a reparative or reactive process. In certain embodiments, fibrosis occurs in response to damage or injury. The term “fibrosis” is to be understood as the formation or development of excess fibrous connective tissue in an organ or tissue as a reparative or reactive process, as opposed to a formation of fibrous tissue as a normal constituent of an organ or tissue.

“Baseline” means measurement of a clinical parameter in a subject just prior to initiation of a treatment. A baseline measurement may be used to confirm that a subject is eligible for treatment with one or more selected pharmaceutical agents. In certain embodiments, a baseline eGFR is obtained from a subject having Alport syndrome, to confirm the subject is eligible for treatment with one or more selected pharmaceutical agents as described herein.

“Short Form 36 Health Survey®” or “SF-36” means a 36 item, subject-reported survey of subject health, used in evaluating subject health status and quality of life. The SF-36 may be used to monitor and compare disease burden of subjects receiving treatment for a disease or condition. The SF-36 includes 8 individual domains: physical functioning, physical role functioning, bodily pain, general health perceptions, vitality, social role functioning, emotional role functioning, and mental health. The SF-36 has been described, for example, by McHorney et al. (Med Care. 1993 March; 31(3):247-63).

“Quality of life” means the extent to which a subject's physical, psychological, and social functioning are impaired by a disease and/or treatment of a disease. Quality of life may be impaired in subjects having a chronic disease, including Alport syndrome.

“Stable dosing regimen” means the amount of a pharmaceutical agent administered to a subject that maintains a therapeutic level of the pharmaceutical agent in the subject. For example, a subject may receive an initial dose of a pharmaceutical agent, which dose may be adjusted higher or lower depending upon how the subject responds to the initial dose. Once the dose providing a desired therapeutic level has been established, the subject is considered to be receiving a stable dosing regimen. The desired therapeutic level may be a desired level of pharmaceutical agent in a tissue (such as blood) of the subject, or a desired pharmacological effect, such as an improvement in one or more symptoms of the disease.

“Slows further progression” means to reduce the rate at which a medical condition moves towards an advanced state.

“Halts further progression” means to stop progression of a medical condition to an advanced state.

“Delay time to dialysis” means to maintain sufficient kidney function such that the need for dialysis treatment is delayed.

“Delay time to renal transplant” means to maintain sufficient kidney function such that the need for a kidney transplant is delayed.

“Improves life expectancy” means to lengthen the life of a subject by treating one or more symptoms of a disease in the subject.

“Anti-miR” means an oligonucleotide having a nucleobase sequence complementary to a microRNA. In certain embodiments, an anti-miR is a modified oligonucleotide.

“Anti-miR-X” where “miR-X” designates a particular microRNA, means an oligonucleotide having a nucleobase sequence complementary to miR-X. In certain embodiments, an anti-miR-X is fully complementary (i.e., 100% complementary) to miR-X. In certain embodiments, an anti-miR-X is at least 80%, at least 85%, at least 90%, or at least 95% complementary to miR-X. In certain embodiments, an anti-miR-X is a modified oligonucleotide.

“miR-21” means the mature miRNA having the nucleobase sequence

(SEQ ID NO: 1)
UAGCUUAUCAGACUGAUGUUGA.

“miR-21 stem-loop sequence” means the stem-loop sequence having the nucleobase sequence

(SEQ ID NO: 2)
UGUCGGGUAGCUUAUCAGACUGAUGUUGACUGUUGAAUCUCAUGGCAACAC
CAGUCGAUGGGCUGUCUGACA.

“Target nucleic acid” means a nucleic acid to which an oligomeric compound is designed to hybridize.

“Targeting” means the process of design and selection of nucleobase sequence that will hybridize to a target nucleic acid.

“Targeted to” means having a nucleobase sequence that will allow hybridization to a target nucleic acid.

“Modulation” means a perturbation of function, amount, or activity. In certain embodiments, modulation means an increase in function, amount, or activity. In certain embodiments, modulation means a decrease in function, amount, or activity.

“Expression” means any functions and steps by which a gene's coded information is converted into structures present and operating in a cell.

“Nucleobase sequence” means the order of contiguous nucleobases in an oligomeric compound or nucleic acid, typically listed in a 5′ to 3′ orientation, independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding.

“Complementary” means that one nucleic acid is capable of hybridizing to another nucleic acid or oligonucleotide. In certain embodiments, complementary refers to an oligonucleotide capable of hybridizing to a target nucleic acid.

“Fully complementary,” also referred to as “100% complementary,” means each nucleobase of an oligonucleotide is capable of pairing with a nucleobase at each corresponding position in a target nucleic acid. In certain embodiments, an oligonucleotide is fully complementary to a microRNA, i.e. each nucleobase of the oligonucleotide is complementary to a nucleobase at a corresponding position in the microRNA. In certain embodiments, an oligonucleotide wherein each nucleobase has complementarity to a nucleobase within a region of a microRNA stem-loop sequence is fully complementary to the microRNA stem-loop sequence.

“Percent complementarity” means the percentage of nucleobases of an oligonucleotide that are complementary to an equal-length portion of a target nucleic acid. Percent complementarity is calculated by dividing the number of nucleobases of the oligonucleotide that are complementary to nucleobases at corresponding positions in the target nucleic acid by the total number of nucleobases in the oligonucleotide.

“Percent identity” means the number of nucleobases in a first nucleic acid that are identical to nucleobases at corresponding positions in a second nucleic acid, divided by the total number of nucleobases in the first nucleic acid. In certain embodiments, the first nucleic acid is a microRNA and the second nucleic acid is a microRNA. In certain embodiments, the first nucleic acid is an oligonucleotide and the second nucleic acid is an oligonucleotide.

“Hybridize” means the annealing of complementary nucleic acids that occurs through nucleobase complementarity.

“Mismatch” means a nucleobase of a first nucleic acid that is not capable of Watson-Crick pairing with a nucleobase at a corresponding position of a second nucleic acid.

“Identical” in the context of nucleobase sequences, means having the same nucleobase sequence, independent of sugar, linkage, and/or nucleobase modifications and independent of the methyl state of any pyrimidines present.

“MicroRNA” means an endogenous non-coding RNA between 18 and 25 nucleobases in length, which is the product of cleavage of a pre-microRNA by the enzyme Dicer. Examples of mature microRNAs are found in the microRNA database known as miRBase (http://microrna.sanger.ac.uk/). In certain embodiments, microRNA is abbreviated as “microRNA” or “miR.”

“microRNA-regulated transcript” means a transcript that is regulated by a microRNA.

“Seed sequence” means a nucleobase sequence comprising from⁶ to 8 contiguous nucleobases of nucleobases 1 to 9 of the 5′-end of a mature microRNA sequence.

“Seed match sequence” means a nucleobase sequence that is complementary to a seed sequence, and is the same length as the seed sequence.

“Oligomeric compound” means a compound that comprises a plurality of linked monomeric subunits. Oligomeric compounds included oligonucleotides.

“Oligonucleotide” means a compound comprising a plurality of linked nucleosides, each of which can be modified or unmodified, independent from one another.

“Naturally occurring internucleoside linkage” means a 3′ to 5′ phosphodiester linkage between nucleosides.

“Natural sugar” means a sugar found in DNA (2′-H) or RNA (2′-OH).

“Internucleoside linkage” means a covalent linkage between adjacent nucleosides.

“Linked nucleosides” means nucleosides joined by a covalent linkage.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside.

“Compound comprising a modified oligonucleotide consisting of” a number of linked nucleosides means a compound that includes a modified oligonucleotide having the specified number of linked nucleosides. Thus, the compound may include additional substituents or conjugates. Unless otherwise indicated, the modified oligonucleotide is not hybridized to a complementary strand hybridized to the modified oligonucleotide, and does not include any additional nucleosides beyond those of the modified oligonucleotide.

“Modified oligonucleotide” means a single-stranded oligonucleotide having one or more modifications relative to a naturally occurring terminus, sugar, nucleobase, and/or internucleoside linkage. A modified oligonucleotide may comprise unmodified nucleosides.

“Modified nucleoside” means a nucleoside having any change from a naturally occurring nucleoside. A modified nucleoside may have a modified sugar and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

“Modified internucleoside linkage” means any change from a naturally occurring internucleoside linkage.

“Phosphorothioate internucleoside linkage” means a linkage between nucleosides where one of the non-bridging atoms is a sulfur atom.

“Modified sugar moiety” means substitution and/or any change from a natural sugar.

“Unmodified nucleobase” means the naturally occurring heterocyclic bases of RNA or DNA: the purine bases adenine (A) and guanine (G), and the pyrimidine bases thymine (T), cytosine (C) (including 5-methylcytosine), and uracil (U).

“5-methylcytosine” means a cytosine comprising a methyl group attached to the 5 position.

“Non-methylated cytosine” means a cytosine that does not have a methyl group attached to the 5 position.

“Modified nucleobase” means any nucleobase that is not an unmodified nucleobase.

“Sugar moiety” means a naturally occurring furanosyl or a modified sugar moiety.

“Modified sugar moiety” means a substituted sugar moiety or a sugar surrogate.

“2′-O-methyl sugar” or “2′-OMe sugar” means a sugar having a O-methyl modification at the 2′ position.

“2′-O-methoxyethyl sugar” or “2′-MOE sugar” means a sugar having a O-methoxyethyl modification at the 2′ position.

“2′-fluoro” or “2′-F” means a sugar having a fluoro modification of the 2′ position.

“Bicyclic sugar moiety” means a modified sugar moiety comprising a 4 to 7 membered ring (including by not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2′-carbon and the 4′-carbon of the furanosyl. Nonlimiting exemplary bicyclic sugar moieties include LNA, ENA, cEt, S-cEt, and R-cEt.

“Locked nucleic acid (LNA) sugar moiety” means a substituted sugar moiety comprising a (CH₂)—O bridge between the 4′ and 2′ furanose ring atoms.

“ENA sugar moiety” means a substituted sugar moiety comprising a (CH₂)₂—O bridge between the 4′ and 2′ furanose ring atoms.

“Constrained ethyl (cEt) sugar moiety” means a substituted sugar moiety comprising a CH(CH₃)—O bridge between the 4′ and the 2′ furanose ring atoms. In certain embodiments, the CH(CH₃)—O bridge is constrained in the S orientation. In certain embodiments, the (CH₂)₂—O is constrained in the R orientation.

“S-cEt sugar moiety” means a substituted sugar moiety comprising an S-constrained CH(CH₃)—O bridge between the 4′ and the 2′ furanose ring atoms.

“R-cEt sugar moiety” means a substituted sugar moiety comprising an R-constrained CH(CH₃)—O bridge between the 4′ and the 2′ furanose ring atoms.

“2′-O-methyl nucleoside” means a 2′-modified nucleoside having a 2′-O-methyl sugar modification.

“2′-O-methoxyethyl nucleoside” means a 2′-modified nucleoside having a 2′-O-methoxyethyl sugar modification. A 2′-O-methoxyethyl nucleoside may comprise a modified or unmodified nucleobase.

“2′-fluoro nucleoside” means a 2′-modified nucleoside having a 2′-fluoro sugar modification. A 2′-fluoro nucleoside may comprise a modified or unmodified nucleobase.

“Bicyclic nucleoside” means a 2′-modified nucleoside having a bicyclic sugar moiety. A bicyclic nucleoside may have a modified or unmodified nucleobase.

“cEt nucleoside” means a nucleoside comprising a cEt sugar moiety. A cEt nucleoside may comprise a modified or unmodified nucleobase.

“S-cEt nucleoside” means a nucleoside comprising an S-cEt sugar moiety.

“R-cEt nucleoside” means a nucleoside comprising an R-cEt sugar moiety.

“β-D-deoxyribonucleoside” means a naturally occurring DNA nucleoside.

“β-D-ribonucleoside” means a naturally occurring RNA nucleoside.

“LNA nucleoside” means a nucleoside comprising a LNA sugar moiety.

“ENA nucleoside” means a nucleoside comprising an ENA sugar moiety.

“Subject” means a human or non-human animal selected for treatment or therapy.

“Subject in need thereof” means a subject that is identified as in need of a therapy or treatment.

“Subject suspected of having” means a subject exhibiting one or more clinical indicators of a disease.

“Administering” means providing a pharmaceutical agent or composition to a subject, and includes, but is not limited to, administering by a medical professional and self-administering.

“Parenteral administration” means administration through injection or infusion. Parenteral administration includes, but is not limited to, subcutaneous administration, intravenous administration, and intramuscular administration.

“Subcutaneous administration” means administration just below the skin.

“Intravenous administration” means administration into a vein.

“Administered concomitantly” refers to the co-administration of two or more agents in any manner in which the pharmacological effects of both are manifest in the subject at the same time.

Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Duration” means the period of time during which an activity or event continues. In certain embodiments, the duration of treatment is the period of time during which doses of a pharmaceutical agent or pharmaceutical composition are administered.

“Therapy” means a disease treatment method. In certain embodiments, therapy includes, but is not limited to, chemotherapy, radiation therapy, or administration of a pharmaceutical agent.

“Treatment” means the application of one or more specific procedures used for the cure or amelioration of a disease. In certain embodiments, the specific procedure is the administration of one or more pharmaceutical agents.

“Ameliorate” means to lessen the severity of at least one indicator of a condition or disease.

In certain embodiments, amelioration includes a delay or slowing in the progression of one or more indicators of a condition or disease. The severity of indicators may be determined by subjective or objective measures which are known to those skilled in the art.

“At risk for developing” means the state in which a subject is predisposed to developing a condition or disease. In certain embodiments, a subject at risk for developing a condition or disease exhibits one or more symptoms of the condition or disease, but does not exhibit a sufficient number of symptoms to be diagnosed with the condition or disease. In certain embodiments, a subject at risk for developing a condition or disease exhibits one or more symptoms of the condition or disease, but to a lesser extent required to be diagnosed with the condition or disease.

“Prevent the onset of” means to prevent the development of a condition or disease in a subject who is at risk for developing the disease or condition. In certain embodiments, a subject at risk for developing the disease or condition receives treatment similar to the treatment received by a subject who already has the disease or condition.

“Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

“Therapeutic agent” means a pharmaceutical agent used for the cure, amelioration or prevention of a disease.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In certain embodiments, a dose is administered as a slow infusion.

“Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

“Therapeutically effective amount” refers to an amount of a pharmaceutical agent that provides a therapeutic benefit to an animal.

“Pharmaceutical composition” means a mixture of substances suitable for administering to an individual that includes a pharmaceutical agent. For example, a pharmaceutical composition may comprise a sterile aqueous solution.

“Pharmaceutical agent” means a substance that provides a therapeutic effect when administered to a subject.

“Active pharmaceutical ingredient” means the substance in a pharmaceutical composition that provides a desired effect.

“Improved organ function” means a change in organ function toward normal limits. In certain embodiments, organ function is assessed by measuring molecules found in a subject's blood or urine.

For example, in certain embodiments, improved kidney function is measured by a reduction in blood urea nitrogen, a reduction in proteinuria, a reduction in albuminuria, etc.

“Acceptable safety profile” means a pattern of side effects that is within clinically acceptable limits.

“Side effect” means a physiological response attributable to a treatment other than desired effects. In certain embodiments, side effects include, without limitation, injection site reactions, liver function test abnormalities, kidney function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

“Subject compliance” means adherence to a recommended or prescribed therapy by a subject.

“Comply” means the adherence with a recommended therapy by a subject.

“Recommended therapy” means a treatment recommended by a medical professional for the treatment, amelioration, or prevention of a disease.

The term “blood” as used herein, encompasses whole blood and blood fractions, such as serum and plasma.

### Overview

Alport syndrome is an inherited form of kidney disease in which an abnormal level of glomerular basement membrane (GBM) is produced, leading to interstitial fibrosis, glomerular sclerosis and typically leads to end-stage renal disease. In the management of Alport syndrome, the primary goal for treatment is to maintain kidney function and prevent the onset of end-stage renal disease (ESRD), which in turn improves life expectancy of subjects with Alport syndrome.

Alport syndrome is characterized by progressive fibrosis due to defects in GBM composition, thus improvements in GBM morphology and kidney function are desirable.

Previously dosage regimens for RG-012, disclosed through clinical trial registries, are fixed doses of 110 mg weekly, and 220 mg weekly. Analysis of pharmacokinetic data from multiple species in preclinical models, as well from healthy volunteers in a multiple ascending dose study, suggested 30 that a weight-based dose of 1.5 mg/kg administered at a less frequent interval of once every two weeks, would be a dose regimen that is both efficacious and suitably safe.

### Certain Modified Oligonucleotides

In certain embodiments, the modified oligonucleotide has the structure 5′-A_{E}C_{S}ATC_{S}AGTC_{S}TGAU_{S}AAGC_{S}TA_{E}-3′ (SEQ ID NO: 3), where nucleosides not followed by a subscript indicate β-D-deoxyribonucleosides; nucleosides followed by a subscript “E” indicate 2′-MOE nucleosides; nucleosides followed by a subscript “S” indicate S-cEt nucleosides; and each internucleoside linkage is a phosphorothioate internucleoside linkage.

In certain embodiments, the modified oligonucleotide has the structure:

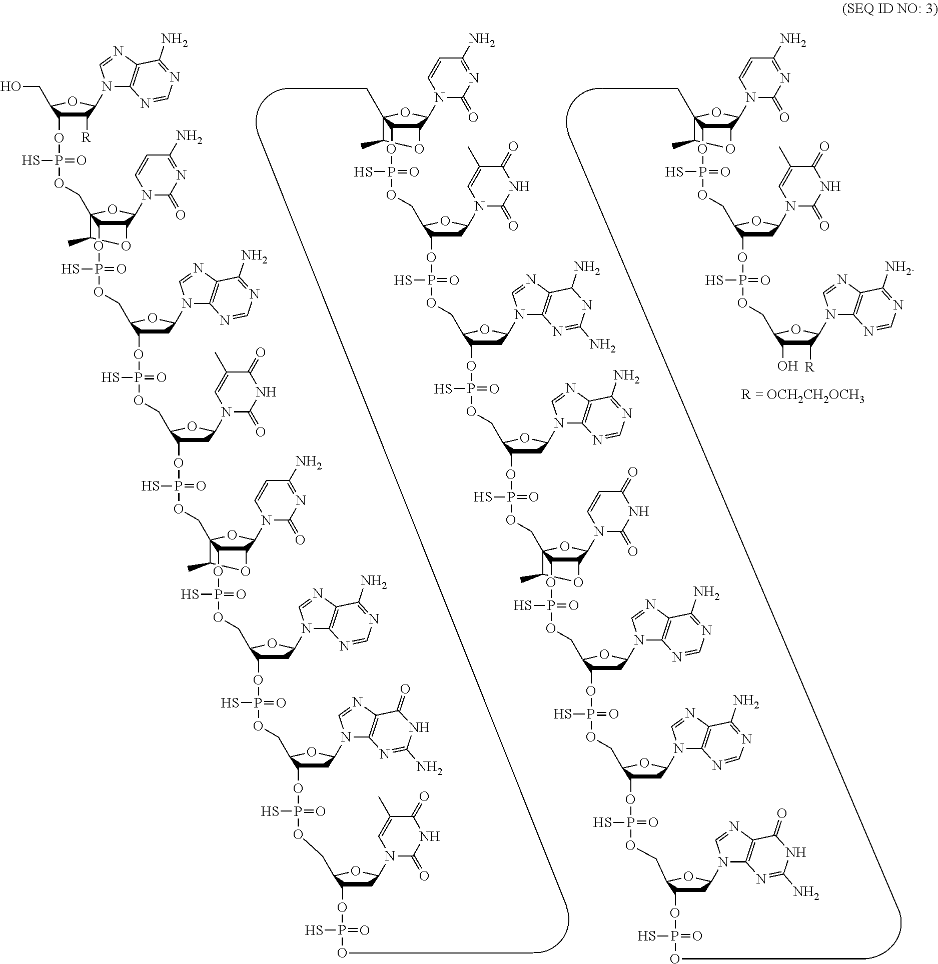

Provided herein are also pharmaceutically acceptable salts of the modified oligonucleotide. Thus, in some embodiments, a modified oligonucleotide has the structure:

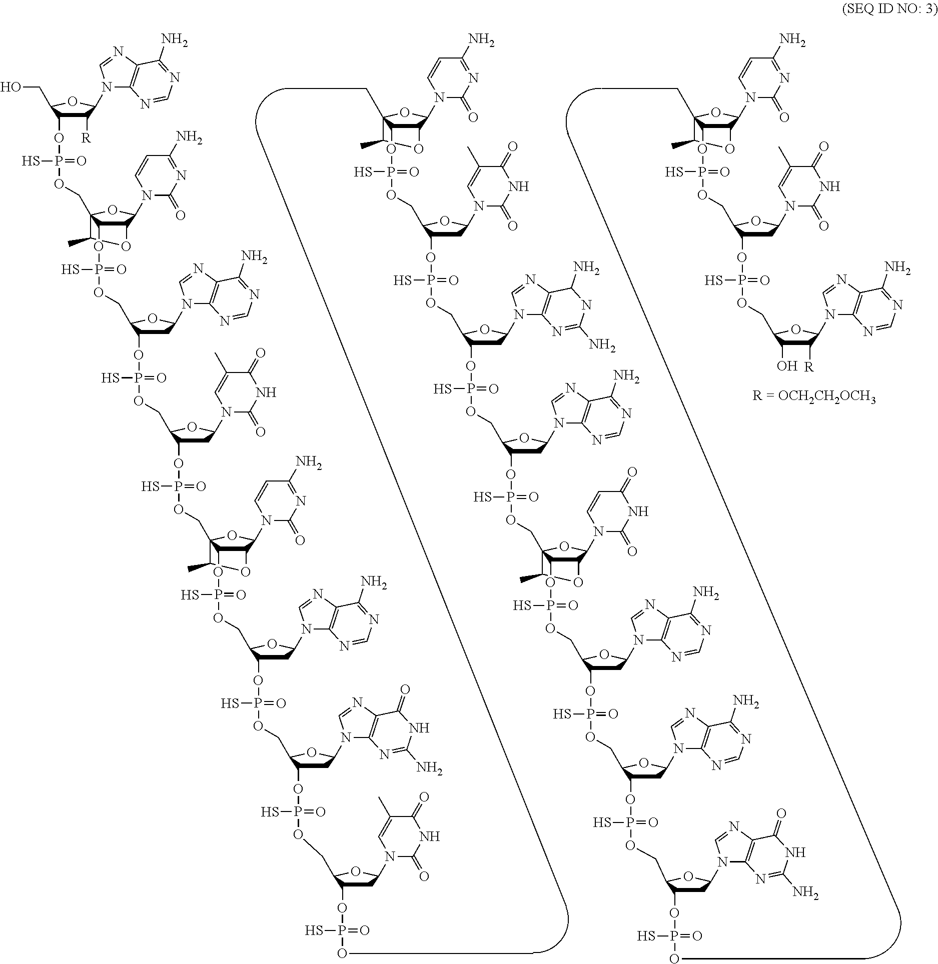

### or a pharmaceutically acceptable salt thereof A nonlimiting exemplary pharmaceutically acceptable salt of the modified oligonucleotide has the structure:

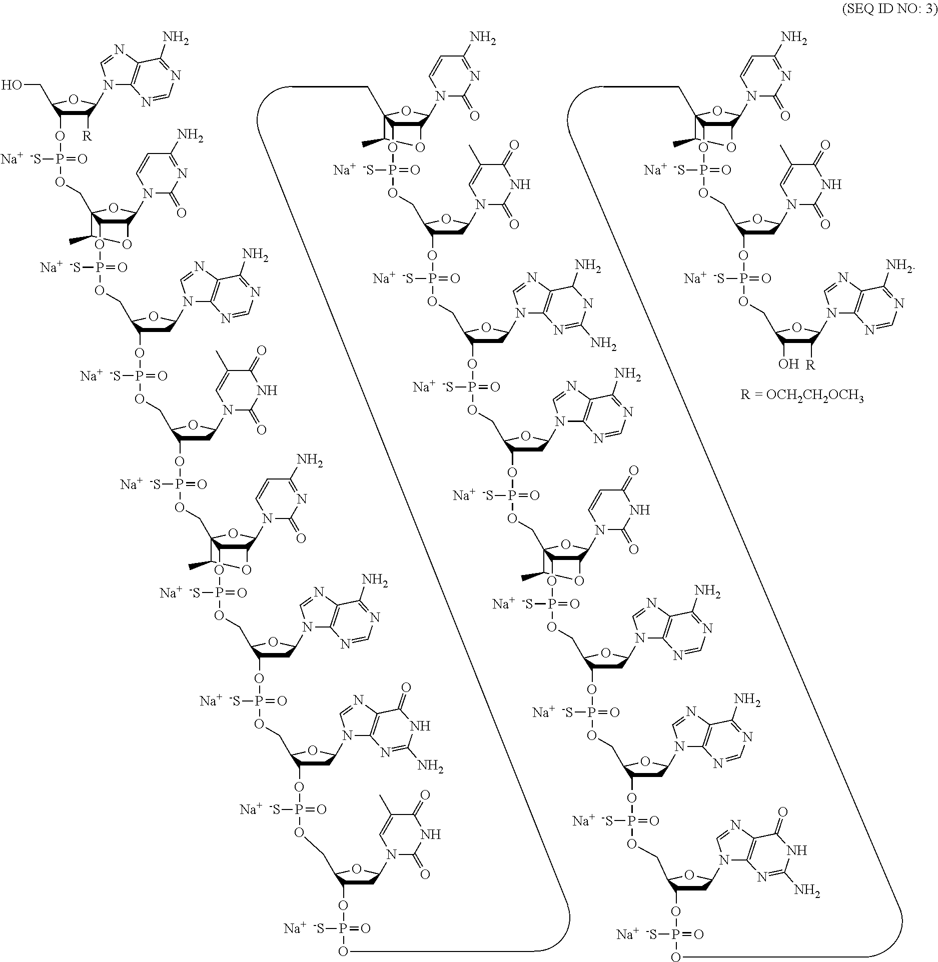

In some embodiments, a pharmaceutically acceptable salt of the modified oligonucleotide comprises fewer cationic counterions (such as Na t) than there are phosphorothioate linkages per molecule (i.e., some phosphorothioate are protonated). In some embodiments, a pharmaceutically acceptable salt of the modified oligonucleotide comprises fewer than 18 cationic counterions (such as Na⁺) per molecule of the modified oligonucleotide. That is, in some embodiments, a pharmaceutically acceptable salt of the modified oligonucleotide may comprise, on average, 1, 2, 3, 4, 5, 6, 7, 8, 9, 10, 11, 12, 13, 14, 15, 16, or 17 cationic counterions per molecule of the modified oligonucleotide, with the remaining phosphorothioate being protonated.

### Certain Uses of the Invention

Provided herein are methods for the treatment of Alport syndrome, comprising administering to a subject having or suspected of having Alport syndrome the modified oligonucleotide provided herein.

In certain embodiments, the subject has been diagnosed as having Alport syndrome prior to administration of the modified oligonucleotide. Diagnosis of Alport syndrome may be achieved through evaluation of parameters including, without limitation, a subject's family history, clinical features (including without limitation proteinuria, albuminuria, hematuria, impaired GFR, for example as determined by measuring eGFR, deafness and/or ocular changes) and results of tissue biopsies. Kidney biopsies may be tested for the presence or absence of the type IV collagen alpha-3, alpha-4, and alpha-5 chains. Additionally, structural changes in the glomerulus can be detected by electron microscopy of kidney biopsy material. A skin biopsy may be tested for the presence of the type IV collagen alpha-5 chain, which is normally present in skin and almost always absent from male subjects with the X-linked form of Alport syndrome. Diagnosis of Alport syndrome may also include screening for mutations in one or more of the Col4a3, Col4a4, or Col4a5 genes.

In certain embodiments, a subject with Alport syndrome has an eGFR of at least 30 20 ml/min/1.73 m² . In certain embodiments, a subject with Alport syndrome has an eGFR of 45 to 90 ml/min/1.73 m² . In certain embodiments, a subject with Alport syndrome has proteinuria of greater equal to or greater than 300 mg protein/g creatinine

In certain embodiments, levels of miR-21 are increased in the kidney of a subject having Alport syndrome. In certain embodiments, prior to administration, a subject is determined to have an increased level of miR-21 in the kidney. miR-21 levels may be measured from kidney biopsy material. In certain embodiments, prior to administration, a subject is determined to have an increased level of miR-21 in the urine or blood of the subject.

In certain embodiments, administration of a modified oligonucleotide complementary to miR-21 results in the improvement of one or more parameters associated with Alport syndrome. In certain embodiments, the administration improves estimated glomerular filtration rate. In certain embodiments, the administration improves measured glomerular filtration rate. In certain embodiments, the administration slows the rate of decline in glomerular filtration rate. In certain embodiments, the administration improves the quality of life of the subject.

In certain embodiments, the administration improves kidney function. In certain embodiments, the administration delays the onset of end-stage renal disease. In certain embodiments, the administration delays time to dialysis. In certain embodiments, the administration delays time to renal transplant. In certain embodiments, the administration improves life expectancy of the subject.

In certain embodiments, the administering reduces kidney fibrosis. In certain embodiments, the administering slows further progression of kidney fibrosis. In certain embodiments, the administration halts further progression of kidney fibrosis. In certain embodiments, the administration reduces hematuria. In certain embodiments, the administration delays the onset of hematuria. In certain embodiments, the administration reduces proteinuria. In certain embodiments, the administration delays the onset of proteinuria.

The subject having or suspected of having Alport syndrome may have a mutation in the gene encoding the alpha 3 chain of type IV collagen (Col4a3), a mutation in the gene encoding the alpha 4 chain of type IV collagen (Col4a4), or a mutation in the gene encoding the alpha 5 chain of type IV collagen (Col4a5). In certain embodiments, the subject is male. In certain embodiments, the subject is female.

In certain embodiments, the subject has impaired kidney function. In certain embodiments, the subject is in need of improved kidney function. In certain embodiments, the subject is identified as having impaired kidney function. In certain embodiments, the subject is identified as having hematuria. In certain embodiments, the subject is identified as having proteinuria.

In any of the embodiments provided herein, a subject may be subjected to certain tests to evaluate kidney function. Such tests include, without limitation, measurement of blood urea nitrogen in the subject; measuring creatinine in the blood of the subject; measuring creatinine clearance in the blood of the subject; measuring proteinuria in the subject; measuring albumin:creatinine ratio in the subject; measuring estimated glomerular filtration rate in the subject; and measuring urinary output in the subject.

In certain embodiments, podocyturia in the subject is assessed by analysis of podocyte numbers and podocyte-specific mRNAs in the urine of the subject.

In any of the embodiments provided herein, proteins present in the urine or blood may be used to evaluate kidney function. Such tests of kidney function include, but are not limited to, measuring N-acetyl-(3-D-glucosaminidase (NAG) protein in the urine of the subject; measuring neutrophil gelatinase-associated lipocalin (NGAL) protein in the urine of the subject; measuring kidney injury molecule-1 (KIM-1) protein in the urine of the subject; measuring interleukin-18 (IL-18) protein in the urine of the subject; measuring connective tissue growth factor (CTGF) levels in the urine of the subject; measuring monocyte chemoattractant protein 1 (MCP1) levels in the urine of the subject; measuring collagen IV (Col IV) fragments in the urine of the subject; measuring collagen III (Col III) fragment levels in the urine of the subject; measuring cystatin C protein in the blood of a subject; measuring β-trace protein (BTP) in the blood of a subject; and measuring 2-microglobulin (B2M) in the blood of a subject. In any of the embodiments provided herein, markers of podocyte injury can be measuring in the urine. Such proteins include nephrin and podocin. The proteins may be quantitated, for example, by enzyme-linked immunosorbent assay (ELISA), or radioimmunoassay (RIA) using commercially available kits.

In any of the embodiments provided herein, the administration of a modified oligonucleotide targeted to miR-21 improves one or more markers of kidney function in the subject. Improvements in markers of kidney function include, without limitation: reduced blood urea nitrogen in the subject; reduced creatinine in the blood of the subject; improved creatinine clearance in the subject; reduced proteinuria in the subject; reduced albumin:creatinine ratio in the subject; improved estimated glomerular filtration rate in the subject; and/or increased urinary output in the subject.

### Certain Pharmaceutical Compositions

Provided herein are pharmaceutical compositions comprising a modified oligonucleotide consisting of 19 linked nucleosides and having the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage; or a pharmaceutically acceptable salt thereof In some such embodiments, the modified oligonucleotide is present in a pharmaceutical composition in its octadecasodium salt form. Unless indicated otherwise, the weights and doses of the modified oligonucleotide 5′-A_{E}C_{S}ATC_{S}AGTC_{S}TGAU_{S}AAGC_{S}TA_{E}-3′ (SEQ ID NO: 3) are based on the weight of the octadecasodium salt form of the modified oligonucleotide. As a nonlimiting example, 110 mg of the octadecasodium salt form of the modified oligonucleotide is equivalent to 103.6 mg of the free acid form of the modified oligonucleotide.

In certain embodiments, a pharmaceutical composition is prepared for injection. In certain embodiments, the pharmaceutical composition prepared for injection comprises the modified oligonucleotide at concentration of 110 mg/mL in a sterile aqueous solution. Suitable routes of administration by injection include subcutaneous and intravenous injection.

In certain embodiments, a pharmaceutical composition provided herein is administered in the form of a bolus dosage unit. In some embodiments, the bolus dosage unit comprises the modified oligonucleotide at a concentration of 110 mg/ml in a sterile aqueous solution. In some embodiments, the bolus dosage unit comprises the modified oligonucleotide at a concentration of 110 mg/ml in a sterile 0.3% sodium chloride aqueous solution. In some embodiments, for a dose of 110 mg of the modified oligonucleotide, a subject is administered a 1 mL bolus dosage unit comprising 110 mg/ml of the modified oligonucleotide. In certain embodiments, the administration is by subcutaneous injection.

In certain embodiments, the modified oligonucleotide is provided as a sterile lyophilized modified oligonucleotide that is reconstituted with a suitable diluent, e.g., aqueous solution, such as water or physiologically compatible buffers such as saline solution, Hank's solution, and Ringer's solution. The reconstituted product may be administered as a subcutaneous injection or as an intravenous infusion. The lyophilized drug product consists of a modified oligonucleotide which has been prepared in a sterile aqueous solution for injection, adjusted to pH 7.0-9.0 with acid or base during preparation, and then lyophilized. The lyophilized drug product may be packaged in a 2 mL Type I, clear glass vial, stoppered with a rubber closure and sealed with an aluminum overseal.

In certain embodiments, the pharmaceutical compositions provided herein may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents.

In certain embodiments, pharmaceutical compositions provided herein one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

### Certain Additional Therapies

Treatments for Alport syndrome may comprise administration of the anti-miR-21 modified oligonucleotide provided herein, and at least one additional therapy. In certain embodiments, the at least one additional therapy comprises a pharmaceutical agent.

In certain embodiments, prior to the administration of the first dose of the modified oligonucleotide, the subject having Alport syndrome has been treated with a stable dosing regimen of an additional therapy for least 30 days. In certain embodiments, the subject is receiving a stable dosing regimen of an angiotensin II receptor blocker. In certain embodiments, the subject is receiving a stable dosing regimen of an angiotensin II converting enzyme inhibitor.

In certain embodiments, the at least one additional therapy comprises a pharmaceutical agent.

In certain embodiments, pharmaceutical agents include angiotensin II receptor blockers (ARB). In certain embodiments, an angiotensin II receptor blocker is candesartan, irbesartan, olmesartan, losartan, valsartan, telmisartan, or eprosartan.

In certain embodiments, pharmaceutical agents include angiotensin II converting enzyme (ACE) inhibitors. In certain embodiments, an ACE inhibitor is captopril, enalapril, lisinopril, benazepril, quinapril, fosinopril, or ramipril.

In certain embodiments, a pharmaceutical agent is an anti-hypertensive agent. Anti-hypertensive agents are used to control blood pressure of the subject.

In certain embodiments, a pharmaceutical agent is a vitamin D analog. Vitamin D analogs may be used to limit the production of parathyroid hormone in the subject.

In certain embodiments, a pharmaceutical agent is an oral phosphate binder that reduces dietary phosphate absorption.

In certain embodiments, pharmaceutical agents include immunosuppressive agents. In certain embodiments, an immunosuppressive agent is a corticosteroid, cyclophosphamide, or mycophenolate mofetil.

In certain embodiments, a pharmaceutical agent is cyclosporine, an HMG-Coenzyme A inhibitor, a vasopeptidase inhibitor, or a TGF-beta-antagonist.

In certain embodiments, an additional therapy is gene therapy. In certain embodiments, the gene therapy provides a normal Col4a3 gene. In certain embodiments, the gene therapy provides a normal Col4a4 gene. In certain embodiments, the gene therapy provides a normal Col4a5 gene.

In certain embodiments, an additional therapy is dialysis. In certain embodiments, an additional therapy is renal transplant.

In certain embodiments, a pharmaceutical agent is an aldosterone antagonsist. In certain embodiments, an aldosterone antagonist is spironolactone.

In certain embodiments, pharmaceutical agents include anti-inflammatory agents. In certain embodiments, an anti-inflammatory agent is a steroidal anti-inflammatory agent. In certain embodiments, a steroid anti-inflammatory agent is a corticosteroid. In certain embodiments, a corticosteroid is prednisone. In certain embodiments, an anti-inflammatory agent is a non-steroidal anti-inflammatory drug. In certain embodiments, a non-steroidal anti-inflammatory agent is ibuprofen, a COX-I inhibitor, or a COX-2 inhibitor.

In certain embodiments, a pharmaceutical agent is a pharmaceutical agent that blocks one or more responses to fibrogenic signals.

In certain embodiments, pharmaceutical agents include anti-diabetic agent. Antidiabetic agents include, but are not limited to, biguanides, glucosidase inhibitors, insulins, sulfonylureas, and thiazolidenediones.

### Certain Modifications

The modified oligonucleotide provided herein comprises sugar-modified nucleosides and modified internucleoside linkages.

A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

Sugar-modified nucleosides include bicyclic sugar moieties. In certain such embodiments, a bicyclic sugar moiety is a D sugar in the alpha configuration. In certain such embodiments, a bicyclic sugar moiety is a D sugar in the beta configuration. In certain such embodiments, a bicyclic sugar moiety is an L sugar in the alpha configuration. In certain such embodiments, a bicyclic sugar moiety is an L sugar in the beta configuration.

Nucleosides comprising such bicyclic sugar moieties are referred to as bicyclic nucleosides or BNAs. In certain embodiments, bicyclic nucleosides include, but are not limited to, (A) α-L-Methyleneoxy (4′-CH₂—O-2′) BNA; (B) β-D-Methyleneoxy (4′-CH₂—O-2′) BNA; (C) Ethyleneoxy (4′-(CH₂)₂—O-2′) BNA; (D) Aminooxy (4′-CH₂—O—N(R)-2′) BNA; (E) Oxyamino (4′-CH₂—N(R)—O-2′) BNA; (F) Methyl(methyleneoxy) (4′-CH(CH₃)—O-2′) BNA (also referred to as constrained ethyl or cEt); (G) methylene-thio (4′-CH₂—S-2′) BNA; (H) methylene-amino (4′-CH₂—N(R)-2′) BNA; (I) methyl carbocyclic (4′-CH₂—CH(CH₃)-2′) BNA; (J) c-MOE (4′-CH₂-OMe-2′) BNA and (K) propylene carbocyclic (4′-(CH₂)₃-2′) BNA as depicted below.

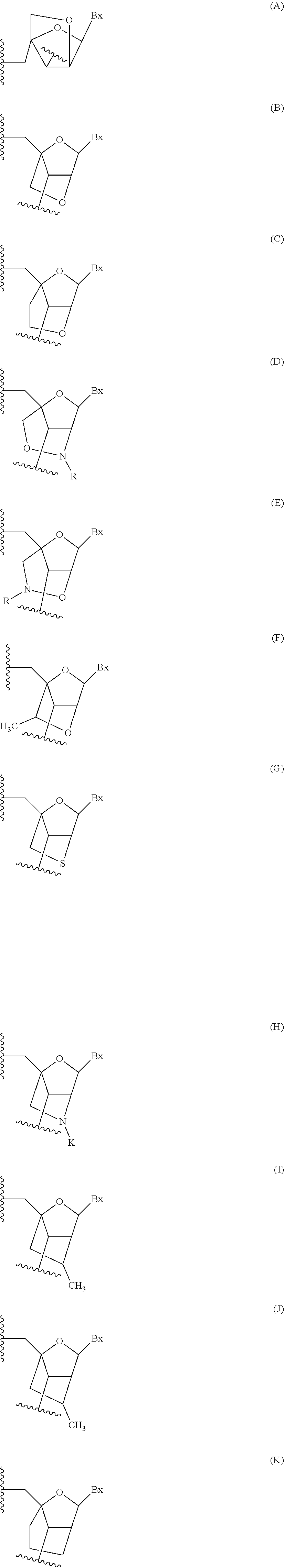

### wherein Bx is a nucleobase moiety and R is, independently, H, a protecting group, or C₁-C₁₂ alkyl.

In certain embodiments, a 2′-modified nucleoside comprises a 2′-substituent group selected from F, OCF₃, O—CH₃, OCH₂CH₂OCH₃, 2′-O(CH₂)₂SCH₃, O—(CH₂)₂—O—N(CH₃)₂, —O(CH₂)₂O(CH₂)₂N—(CH₃)₂, and 0-CH₂—C(═O)—N(H)CH₃.

In certain embodiments, a 2′-modified nucleoside comprises a 2′-substituent group selected from F, O—CH₃, and OCH₂CH₂OCH₃.

In certain embodiments, a sugar-modified nucleoside is a 4′-thio modified nucleoside. In certain embodiments, a sugar-modified nucleoside is a 4′-thio-2′-modified nucleoside. A 4′-thio modified nucleoside has a β-D-ribonucleoside where the 4′-0 replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-O—(CH₂)₂-OCH₃, and 2′-F.

In certain embodiments, a modified oligonucleotide comprises one or more internucleoside modifications. In certain such embodiments, each internucleoside linkage of a modified oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In certain embodiments, each internucleoside linkage of a modified oligonucleotide is a phosphorothioate internucleoside linkage.

### Certain Kits

The present invention also provides kits. In some embodiments, the kits comprise the anti-miR-21 modified oligonucleotide provided herein. In some embodiments, the modified oligonucleotide can be present within a vial. A plurality of vials, such as 10, can be present in, for example, dispensing packs. In some embodiments, the vial is manufactured so as to be accessible with a syringe. The kit can also contain instructions for using the modified oligonucleotide.

In some embodiments, the kits may be used for administration of the modified oligonucleotide. In such instances, in addition to the modified oligonucleotide, the kit can further comprise one or more of the following: syringe, alcohol swab, cotton ball, and/or gauze pad. In some embodiments, the modified oligonucleotide can be present in a pre-filled syringe (such as a single-dose syringes with, for example, a 27 gauge, ½ inch needle with a needle guard), rather than in a vial. A plurality of pre-filled syringes, such as 10, can be present in, for example, dispensing packs. The kit can also contain instructions for administering the modified oligonucleotide.

### Certain Experimental Models

In certain embodiments, the present invention provides methods of using and/or testing modified oligonucleotides of the present invention in an experimental model. Those having skill in the art are able to select and modify the protocols for such experimental models to evaluate a pharmaceutical agent of the invention.

Generally, modified oligonucleotides are first tested in cultured cells. Suitable cell types include those that are related to the cell type to which delivery of a modified oligonucleotide is desired in vivo. For example, suitable cell types for the study of the methods described herein include primary or cultured cells.

In certain embodiments, the extent to which the modified oligonucleotide interferes with the activity of miR-21 is assessed in cultured cells. In certain embodiments, inhibition of miR-21 activity may be assessed by measuring the levels of miR-21 in a cell or tissue. Alternatively, the level of a predicted or validated microRNA-regulated transcript may be measured. An inhibition of miR-21 activity may result in the increase in the miR-21-regulated transcript, and/or the protein encoded by miR-21-regulated transcript. Further, in certain embodiments, certain phenotypic outcomes may be measured.

Several animal models are available to the skilled artisan for the study of miR-21 in models of human disease. For example, inhibitors of miR-21 may be studied in an experimental model of Alport syndrome, for example Col4a3 knockout mice (Col4a3^{-/-} mice). The severity of the disease in the mouse model depends upon the genetic background of the mouse carrying the Col4a3 mutation. For example, the onset and progression of the disease are generally more rapid on the 129X1/SvJ relative to the C57BL/6J background. Accordingly, the genetic background of the Col4a3^{-/-} mouse may be selected to vary the onset and progression of disease. Additional models include canine models of X-linked, autosomal recessive or autosomal dominant Alport syndrome. See, for example, Kashtan, Nephrol. Dial. Transplant, 2002, 17: 1359-1361.

### Certain Quantitation Assays

The effects of antisense inhibition of miR-21 following the administration a modified oligonucleotide may be assessed by a variety of methods known in the art. In certain embodiments, these methods are used to quantitate microRNA levels in cells or tissues in vitro or in vivo. In certain embodiments, changes in microRNA levels are measured by microarray analysis. In certain embodiments, changes in microRNA levels are measured by one of several commercially available PCR assays, such as the TaqMan® MicroRNA Assay (Applied Biosystems). In certain embodiments, antisense inhibition of miR-21 is assessed by measuring the mRNA and/or protein level of a target of miR-21. Antisense inhibition of miR-21 generally results in the increase in the level of mRNA and/or protein of a target of the microRNA.

### Target Engagement Assay

Modulation of microRNA activity with an anti-miR or microRNA mimic may be assessed by measuring target engagement. In certain embodiments, target engagement is measured by microarray profiling of mRNAs. The sequences of the mRNAs that are modulated (either increased or decreased) by the anti-miR or microRNA mimic are searched for microRNA seed sequences, to compare modulation of mRNAs that are targets of the microRNA to modulation of mRNAs that are not targets of the microRNA. In this manner, the interaction of the anti-miR with miR-21, or miR-21 mimic with its targets, can be evaluated. In the case of an anti-miR, mRNAs whose expression levels are increased are screened for the mRNA sequences that comprise a seed match to the microRNA to which the anti-miR is complementary.

### EXAMPLES

The following examples are presented in order to more fully illustrate some embodiments of the invention. They should in no way be construed, however, as limiting the broad scope of the invention.

Those of ordinary skill in the art will readily adopt the underlying principles of this discovery to design various compounds without departing from the spirit of the current invention.

### Example 1

### A Phase 2 Study in Subjects with Alport Syndrome

The study is a randomized, double-blind, placebo-controlled, multi-center, Phase 2 study conducted in subjects with Alport syndrome at multiple investigative centers. Alport syndrome is an inherited form of kidney disease caused by mutations in genes coding for the capillary basement membrane collagen IV. Over time, Alport syndrome causes damage to the kidneys. The study drug is RG-012. Previously dosage regimens for RG-012, disclosed through clinical trial registries, are fixed doses of 110 mg weekly, and 220 mg weekly. Analysis of pharmacokinetic data from multiple species in preclinical models, as well from healthy volunteers in a multiple ascending dose study, suggested that a weight-based dose of 1.5 mg/kg administered at a less frequent interval of once every two weeks, would be a dose regimen that is both efficacious and suitably safe.

The active ingredient (AI) in RG-012 is the octadecasodium salt of a 19-base, single-stranded, chemically modified oligonucleotide of the structure:
5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage.

RG-012 is formulated as an aqueous solution of the AI containing 0.3% sodium chloride and is administered by the subcutaneous (SC) route once every two weeks.

In some instances, the major active metabolite (AM) lacks the 3′-terminal 2′-MOE modified “A” nucleoside:
5′-A_{E}C_{S}ATC_{S}AGTC_{S}TGAU_{S}AAGC_{S}T-3′ (SEQ ID NO: 4), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage.

The primary objective is to assess the safety and tolerability of RG-012 when administered weekly for 48 weeks. Safety and tolerability are assessed by variables such as adverse events, laboratory parameters, vital signs, ECGs, and injection site reactions.

Secondary objectives include:
Assessment of the effect of RG-012 on other selected blood, urine, and renal biomarkers;
Assessment of the pharmacokinetic (PK) parameters of the parent compound (AI) and its active major metabolite (AM) following administration of RG-012;
Assessment of the potential formation of anti-drug antibodies (ADAs) following administration of RG-012;
Assessment of the preliminary efficacy of RG-012 to reduce the decline in renal function over time;
Assessment in quality of life (QoL) following administration of RG-012, using the Short Form 36 Health Survey®.

| Study Arms | Assigned Interventions |
|---|---|
| Placebo Comparator: Placebo | Drug: Placebo |
| 1 mL, weekly, 48 weeks | 2 μg/mL riboflavin in 0.9% sodium |
| | chloride |
| Experimental: | Drug: RG-012 for subcutaneous |
| 1.5 mg/kg RG-012, every | injection, supplied ready for |
| two weeks, for 48 weeks | injection as a formulation of |
| | Active Ingredient in 0.3% |
| | sodium chloride, at a nominal |
| | concentration of 110 mg/mL |

Double-Blind, Placebo-Controlled Treatment Period - Eligible subjects are randomized in a 1:1:1 ratio to receive biweekly (every two weeks) subcutaneous (SC) injections of RG-012 1.5 mg/kg, or placebo, for 48 weeks.

The investigator may titrate an individual subject's biweekly RG-012 dose down to 0.75 mg/kg based on tolerability. Subjects whose biweekly RG-012 doses have been reduced to 0.75 mg/kg may also have their doses readjusted back to 1.5 mg/kg based on Investigator judgment.

Subjects taking angiotensin converting enzyme (ACE) inhibitors or angiotensin II receptor blockers (ARBs) maintain these agents at a stable dose and regimen for the duration of the active treatment period. All other concomitant medications are also maintained at a stable dosing regimen during the study.

Open Label Extension Period—Subjects completing 48 weeks of treatment are eligible to screen for enrollment in a 48-week extension study in which all subjects receive active treatment.

Follow-up Period—Upon completion of study treatment, all subjects will enter a 12-week post-treatment follow-up period, which will include a brief home visit at Follow-up Week 2, a home visit at Follow-up Week 4, and a site visit at Follow-up Week 12. Subjects who complete the double-blind, placebo-controlled treatment period but do not enter the open-label treatment extension period will enter directly into the post-treatment follow-up period.

Inclusion Criteria:
1. Male subjects aged 18 to 65 years (inclusive)
2. Confirmed diagnosis of Alport syndrome (clinical, histopathologic, and/or genetic diagnosis)
3. As estimated with the Chronic Kidney Disease Epidemiology Collaboration (CKD-EPI) creatinine or creatinine-cystatin C equation, the following eGFR criteria must be met:
   a. eGFR at the initial screening measurement between 40 and 90 ml/min/1.73 m²;
   b. a decline in eGFR of ≥5 mL/min/1.73 m²/year based on a linear regression slope analysis of ≥4 eGFR measurements from the previous 52 weeks. A decline in eGFR of ≥5 mL/min/1.73 m²/year is equivalent to an eGFR slope of ≤−5 mL/min/1.73 m²/year. eGFR slope, or the change in a subject's eGFR over time, is calculated using linear regression.
4. Proteinuria ≥300 mg protein/g creatinine at initial screening and baseline visits

Subjects without a sufficient number of prior eGFR measurements to allow for the calculation of eGFR slope may qualify for the study if they are male, diagnosed with XLAS, and 18 to 30 years of age (inclusive).

Subjects taking an ACE inhibitor and/or an ARB must be on a stable dosing regimen of an ACE inhibitor and/or ARB for ≥30 days prior to screening.

Exclusion Criteria:
1. Causes of chronic kidney disease aside from Alport syndrome (including, but not limited to, diabetic nephropathy, hypertensive nephropathy, lupus, IgA nephropathy).
2. ESRD as evidenced by ongoing dialysis therapy or history of renal transplantation.
3. Any other condition or circumstance that, in the opinion of the responsible clinical investigator, may make the subject unlikely to complete the study or comply with study procedures and requirements, or may pose a risk to the subject's safety and well-being.

Pharmacodynamic endpoints include change over time in pharmacodynamic and biomarker endpoints including:
blood urea nitrogen [BUN];
protein/albumin ratio and albumin/creatinine ratio in urine;
creatinine, cystatin C, kidney injury molecule-1 [KIM-1], β-2 microglobulin, and clusterin in both serum and urine;
asymmetric dimethylarginine (ADMA), transforming growth factor-β (TGFβ), connective tissue growth factor (CTGF), and neutrophil gelatinase-associated lipocalin (NGAL) in both serum and urine;
granulocyvte-macrophage colony-stimulating factor (GM-CSF), monocyte chemoattractant protein-1 (MCP-1), calbindin, interleukin-18 (IL-18), and epidermal growth factor (EGF) in urine;
and podocyturia as measured by analysis of podocyte numbers and podocyte-specific mRNAs in urine.

Pharmacokinetic endpoints include plasma concentrations and calculated PK parameters of the parent compound (AI) and its active metabolite (AM).

Efficacy endpoints include:
Linear regression slope of estimated glomerular filtration rate (eGFR) from baseline to Weeks 24, 48, and 96;
Absolute and percent change in eGFR values from baseline to Weeks 24, 48, and 96;
Proportion of subjects with a response to treatment from baseline to Weeks 24, 48, and 96 based on the following definitions:
   eGFR slope>-2 mL/min/1.73m²/year
   eGFR slope>-5 mL/min/1.73m²/year
   eGFR slope>-10 mL/min/1.73m²/year
   eGFR slope>-15 mL/min/1.73m²/year;
Proportion of subjects with a response to treatment from baseline to Weeks 24, 48, and 96 based on the following definitions:
   <5% reduction in eGFR value relative to baseline
   <10% reduction in eGFR value relative to baseline
   <20% reduction in eGFR value relative to baseline
   <30% reduction in eGFR value relative to baseline;
Difference in eGFR slope between the screening period and the treatment period;
Number and proportion of subjects who reach end stage renal disease (ESRD) as defined by an eGFR ≤15 mUmin/1.73m² or initiation of hemodialysis or renal transplantation; and
Change over time in QoL as measured using the Short Form 36 Health Survey® (SF-36).

It is anticipated that a dose of 1.5 mg/kg of RG-012, administered once every two weeks to a subject having Alport syndrome, provides an appropriate safety margin, and results in improved efficacy endpoints, for example, maintenance of or improvement in kidney function.

### Example 2

### Calculation of glomerular filtration rate

The National Kidney Foundation provides several equations, developed by the Chronic Kidney Disease Epidemiology Collaboration (CKD-EPI) to calculate estimated glomerular filtration rate (eGFR) in a subject. One or more of these equations is used to measure eGFR in subjects screened for and participating in the Phase 2 study described herein.

### CKD-EPI Creatinine Equation (2009)

Expressed as a single equation: eGFR=141×min(Scr/κ, 1)^{a}×max(S_{Cr}/κ, 1)^{-1.209}×0.993^{Age}×1.018 [if female]×1.159 [if African-American]

Abbreviations/Units
eGFR (estimated glomerular filtration rate)=mL/min/1.73 m²
S_{Cr} (serum creatinine)=mg/dL
S_{cys} (standardized serum cystatin C)=mg/l
κ=0.7 (females) or 0.9 (males)
α=−0.248 (females) or −0.207 (males)
min(S_{Cr}/κ or 1)=indicates the minimum of S_{Cr}/κ or 1
max(S_{Cr}/κ or 1)=indicates the maximum of S_{Cr}/κ or 1
min(S_{cys}/0.8, 1)=indicates the minimum of S_{cys}/0.8, 1
max(S_{cys}/0.8, 1)=indicates the maximum of S_{cys}/0.8, 1
age=years.

### CKD-EPI Creatinine-Cystatin Equation (2012)

Expressed as a single equation: eGFR=135×min(S_{Cr}/κ, 1)^{α}×max(S_{Cr}/κ, 1)^{-0.601}×min(S_{cys}/0.8, 1)^{-0.375} max(S_{cys}/0.8, 1)^{-0.711}×0.995^{Age}×0.969 [if female]×1.08 [if African-American]

Abbreviations/Units
eGFR (estimated glomerular filtration rate)=mL/min/1.73 m²
S_{Cr} (standardized serum creatinine)=mg/dL
κ=0.7 (females) or 0.9 (males)
α=−0.329 (females) or −0.411 (males)
min=indicates the minimum of Scr/κor 1
max=indicates the maximum of S_{Cr}/κ or 1
age=years.

### Example 3

### A Phase 1 Study with Biopsy

The study is Phase 1 study of the safety, pharmacodynamics, and pharmacokinetics of RG-012 administered to subjects with Alport syndrome. During this open-label study, all eligible subjects will receive RG-012. The study consists of two parts (Part A and Part B). During Part A, half of the participants will receive a single dose of RG-012 and half will receive 4 doses of RG-012 (one dose every other week for 6 weeks). All subjects will undergo two renal biopsies, one before and one after receiving RG-012, to assess the effect of RG-012 on the kidney. After completing Part A, subjects will be able to enter Part B of the study. During Part B, all subjects will receive RG-012 every other week for 48 weeks.

| | Assigned |
|---|---|
| Study Arms | Interventions |
| Experimental: RG-012 Single Dose | Drug: RG-012 |
| 1.5 mg/kg RG012 subcutaneous injection | RG-012 in 0.3% |
| | sodium chloride |
| Experimental: RG-012 Every Other Week | Drug: RG-012 |
| 1.5 mg/kg RG-012 subcutaneous injections | RG-012 in 0.3% |
| every other week | sodium chloride |

Primary Outcome Measures include:
Incidence and severity of adverse events; and
Effect of RG-012 on renal miR-21.

Secondary Outcome Measures include:
Pharmacokinetic (PK) parameter—Cmax (maximum observed plasma concentration)
Pharmacokinetic (PK) parameter—Tmax (time to maximum observed plasma concentration)
Pharmacokinetic (PK) parameter—AUC (area under the plasma concentration vs. time curve)

Inclusion Criteria:
1. Males or females, ages 18 to 65 years
2. Confirmed diagnosis of Alport syndrome
3. eGFR between 40 and 90 mL/min/1.73m²
4. Proteinuria of at least 300 mg protein/g creatinine
5. For subjects taking an ACE inhibitor or an ARB, the dosing regimen should be stable for at least 30 days prior to screening.
6. Willing to comply with contraception requirements.

Exclusion Criteria:
1. Causes of chronic kidney disease aside from Alport syndrome (such as diabetic nephropathy, hypertensive nephropathy, lupus nephritis, or IgA nephropathy)
2. End stage renal disease (ESRD) as evidenced by ongoing dialysis therapy or history of renal transportation
3. Any other condition that may pose a risk to the subject's safety and well-being
4. Female subjects who are pregnant or lactating

Primary Outcome Measures include:
Incidence and severity of adverse events; and
Effect of RG-012 on renal miR-21.

Secondary Outcome Measures include:
Pharmacokinetic (PK) parameter—Cmax (maximum observed plasma concentration)
Pharmacokinetic (PK) parameter—Tmax (time to maximum observed plasma concentration)
Pharmacokinetic (PK) parameter—AUC (area under the plasma concentration vs. time curve)

In this study, subjects undergo a kidney biopsy before the first dose of RG-012, and after the final dose of RG-012. Blood samples are collected. RNA is isolated from the kidney, and miR-21 levels are measured. RG-012 levels are measured in blood and kidney tissue. miR-21-regulated mRNA transcripts may be measured.

It is anticipated that a dose of 1.5 mg/kg once every two weeks, provides an sufficient levels of RG-012 in the kidney to engage (i.e., inhibit) miR-21.

1. A method for treating Alport syndrome comprising administering to a subject having Alport syndrome two or more doses of a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, and wherein a dose of 1.5 mg/kg is administered at a frequency of two weeks between doses.

2. The method of claim 1, wherein the dose is delivered in a pharmaceutically acceptable diluent.

3. The method of claim 2, wherein the pharmaceutically acceptable diluent is a saline solution.

4. The method of claim 3, wherein the saline solution is a 0.3% sodium chloride solution.

5. The method of claim 1, wherein the concentration of the modified oligonucleotide in the pharmaceutically acceptable diluent is at least 110 mg/mL.

6. The method of claim 1, wherein the dose is a single bolus injection of 110 mg/mL of the modified oligonucleotide.

7. The method of claim 1, wherein the pharmaceutical composition is administered as a subcutaneous injection.

8. The method of claim 7, wherein the subcutaneous injection is administered in the anterior abdominal wall of the subject.

9. The method of claim 1, comprising selecting a subject who has been diagnosed with Alport syndrome by clinical, histopathologic, and/or genetic criteria.

10. The method of claim 1, wherein the subject has an estimated glomerular filtration rate of 30 ml/min/1.73 m² prior to receiving the first dose of the modified oligonucleotide.

11. The method of claim 9, wherein the subject has an estimated glomerular filtration rate (eGFR) between 45 and 90 ml/min/1.73 m² prior to receiving the first dose of the modified oligonucleotide.

12. The method of claim 9, wherein the estimated glomerular filtration rate of the subject is declining at a rate ml/min/1.73 m²/year prior to receiving the first dose of the modified oligonucleotide.

13. The method of claim 9, wherein the subject is male, has been diagnosed with X-linked Alport syndrome, and is between 18 and 30 years of age.

14. The method of claim 9, wherein the subject has proteinuria of greater than 300 milligrams of protein per gram of creatinine prior to receiving the first dose of the modified oligonucleotide.

15. The method of claim 1, wherein the subject, following administration of the modified oligonucleotide, experiences an improvement in one or more parameters associated with Alport syndrome selected from the group consisting of:
a. estimated glomerular filtration rate;
b. rate of decline in estimated glomerular filtration rate; and
c. quality of life using the Short Form 36 Health Survey®.

16. The method of claim 1, wherein the subject, following administration of the modified oligonucleotide, exhibits an improvement in one or more renal biomarkers selected from the group consisting of:
a. miR-21 in biopsy tissue;
b. blood urea nitrogen;
c. urine protein/albumin ratio;
d. urine albumin/creatine ratio;
e. creatinine;
f. urine podocyturia;
g. kidney injury molecule-1;
h. beta-2 microglobulin;
i. clusterin;
j. cystatin C;
k. asymmetric dimethylarginine;
l. transforming growth factor-beta;
m. connective tissue growth factor; and
n. neutrophil gelatinase-associated lipocalin.

17. The method of claim 16, wherein one or more of creatinine, cystatin C, kidney injury molecule-1, beta-2 microglobulin, and/or clusterin is measured in a blood sample of the subject.

18. The method of claim 16, wherein one or more of creatinine, cystatin C, kidney injury molecule-1, beta-2 microglobulin, and/or clusterin is measured in a urine sample of the subject.

19. (canceled)

20. (canceled)

21. (canceled)

22. (canceled)

23. (canceled)

24. A method for treating Alport syndrome in a subject, the method comprising:
a. selecting a subject who has been diagnosed with Alport syndrome using clinical, histopathologic, and/or genetic criteria;
b. administering to the subject two or more doses of a pharmaceutical composition comprising a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, wherein the dose of the modified oligonucleotide is 1.5 mg/kg and wherein the doses are administered with a frequency of two weeks between doses,
c. wherein the subject, following administration of the pharmaceutical composition, exhibits an improvement in one or more AS associated parameters selected from:
i. estimated glomerular filtration rate (eGFR);
ii. rate of decline of eGFR; and
iii. quality of life (QOL) as measured by the Short Form 36 Health Survey®.

25. (canceled)

26. A method for reducing decline in renal function over time in a subject with Alport syndrome, the method comprising:
a. selecting a subject diagnosed with Alport syndrome confirmed by clinical, histopathologic, and/or genetic criteria, wherein the subject has:
i. an estimated glomerular filtration rate of at least 30 ml/min/1.73 m²;
ii. a decline in the rate of estimated glomerular filtration rate of ml/min/1.73 m²/year;
iii. proteinuria greater equal to or greater than 300 mg protein/g creatinine; and
iv. been treated with a stable dosing regimen of an ACE inhibitor and/or an ARB for at least 30 days;
b. administering to the subject two or more doses of a pharmaceutical composition comprising a modified oligonucleotide, wherein the modified oligonucleotide consists of 19 linked nucleosides and has the structure 5′-A_{E}C_{S}ATC_{S}AGTC_{S}TGAU_{S}AAGC_{S}TA_{E}-3′ (SEQ ID NO: 3), where nucleosides not followed by a subscript are β-D-deoxyribonucleosides; nucleosides followed by a subscript “E” are 2′-MOE nucleosides; nucleosides followed by a subscript “S” are S-cEt nucleosides, and each internucleoside linkage is a phosphorothioate internucleoside linkage, wherein the dose of the modified oligonucleotide is 1.5 mg/kg and wherein the doses are administered with a frequency of two weeks between doses,
c. wherein the subject, following administration of the pharmaceutical composition, exhibits an improvement in one or more Alport syndrome associated parameters selected from:
i. estimated glomerular filtration rate (eGFR);
ii. rate of decline of eGFR; and
iii. quality of life (QOL) as measured by the Short Form 36 Health Survey®.

27. (canceled)

28. (canceled)

29. (canceled)

30. (canceled)

31. (canceled)

32. (canceled)
